(19) United States
(12) Patent Application Publication
(10) Pub. No.: US 2022/0054720 A1
(43) Pub. Date: Feb. 24, 2022

(54) ADAPTABLE SUCTION TUBING AND LIGHT APPARATUS FOR SURGICAL RETRACTORS

(71) Applicants: Hamid C. Hajarian, Aliso Viejo, CA (US); Vahid Hamidi, Aliso Viejo, CA (US)

(72) Inventors: Hamid C. Hajarian, Aliso Viejo, CA (US); Vahid Hamidi, Aliso Viejo, CA (US)

(21) Appl. No.: 16/998,177

(22) Filed: Aug. 20, 2020

(51) Int. Cl. A61M 1/00; A61B 17/02; A61B 90/30

(52) U.S. Cl. CPC A61M 1/0086; A61B 17/02; A61B 2217/005; A61B 2090/306; A61B 90/30

(57) ABSTRACT

An adaptable suction tubing apparatus for a surgical retractor is disclosed. The suction tubing apparatus comprises a suction tip connector, an extruded rectangular tubing and an end suction connector. All components also have side protrusions or bumps for nesting into top channels over the top surface of a surgical retractor. These side protrusions provides snug fit within a surgical retractor. Optionally the tubing can have double sided adhesive tape for placement over the top surface of a standard surgical retractor. The suction tubing apparatus is designed for one time use and has low profile and conformed to the contour of any retractors perfectly.

### FIELD OF THE INVENTION

The present invention relates to surgical retractor that is used by a surgeon in surgery. More particularly, the surgical retractor uses an adaptable suction and light overly that is flexible and conformed to the contour of a retractor.

### BACKGROUND OF THE INVENTION

There are many types of surgical retractors used today in surgical field. Some of these retractors have suction and light, and some are just simple plain retractors. Those retractors with suction and light are having pre-formed tubular jackets over the instrument for providing suction and light and cannot be removed or disassembled from instrument for better sterilization processes. Normally the tubular jacket are made of stainless steel material and it is spot welded to the instrument. Furthermore the function of the suction tube is very limited and can only evacuate the smoke from the surgical field. In most cases when surgeon is performing an operation, he or she needs another suction device to evacuate the fluid from the field in order to visually examine and evaluate the surgical field.

However, this invention overcomes the shortcomings of prior art by using a suction tubing device to be placed right over a surgical retractor and can easily be removed or peel off after usage. The surgeon has options to place the suction tubing anywhere over the retractor instrument. He or she can use it for smoke evacuation or for fluid evacuations as needed for the surgical procedures. The suction and light tubing apparatus specifically designed for one time use and therefor it is disposable and no need for re-sterilization process.

### SUMMARY OF THE INVENTION

An adaptable suction tubing apparatus for a surgical retractor is disclosed. The suction tubing apparatus comprises a suction tip connector, an extruded rectangular tubing and an end suction connector. All components have side protrusions or bumps for nesting over the top surface of a surgical retractor channels. These side protrusions provides snug fit with a surgical retractor. The suction tubing apparatus is designed for one time use and has low profile and conformed to the contour of any retractors perfectly.

It is object of present invention to have a double sided adhesive tape on the bottom surface of the tubing apparatus for the placement over the top surface of a standard surgical retractor.

It is also object of present invention to have combination of suction and fiber optic light for this apparatus. Furthermore the fiber optics lights has universal end connector and can be used with many different light source machines.

It is yet another object of the present invention to configure at least one dedicated lumen in suction strip for the fiber optic adjacent to suction lumen or within suction lumen.

Further objects and advantages of this invention will become apparent from consideration of the drawings and descriptions that follow.

### BRIEF DESCRIPTION OF THE DRAWINGS

The drawings constitute a part of this specification and include exemplary embodiments to the invention, which may be portrayed in various forms. It is to be understood that in some instances various aspects of the invention may be exaggerated or enlarged to facilitate an understanding of the invention.

FIG. 1 is a perspective view showing the suction apparatus snug into surgical retractor showing the present invention.

FIG. 2 rear perspective view of FIG. 1.

FIG. 3 is an exploded view of surgical retractor and suction apparatus assembly.

FIG. 4 is an exploded view of suction apparatus.

FIG. 5 is a perspective view of suction tip for suction apparatus.

FIG. 6 is an assembled cross sectional taken along line 6-6 in FIG. 2.

FIG. 7 is a cross sectional view taken along line 7-7 in FIG. 6.

FIG. 8 is a cross sectional view similar to FIG. 7 but showing is exploded view.

FIG. 9 is a perspective view of a modified surgical retractor in conjunction with suction apparatus.

FIG. 10 is a cross sectional view taken along line 10-10 in FIG. 9.

FIG. 11 is a perspective view showing a modified version of suction apparatus but having light assembly.

FIG. 12 is an exploded view of FIG. 11.

FIG. 13 is a cross sectional view taken along line 13-13 in FIG. 11.

FIG. 14 is a cross sectional view similar to FIG. 13 but showing the fiber optic light inside the suction strip lumen.

FIG. 15 is a cross sectional view similar to FIG. 13 but showing the fiber optic lights embedded into lower portion of the suction strip.

FIG. 16 is a cross sectional view showing an alternate embodiment of suction strip having at least one suction lumen and at least one fiber optic lumen.

FIG. 18 is perspective view showing suction apparatus having double sided adhesive for purpose of attaching to standard surgical retractor.

FIG. 18 is perspective view showing suction and light apparatus having double sided adhesive for purpose of attaching to standard surgical retractor

FIG. 19 is a perspective view of suction and light apparatus in conjunction with a malleable retractor.

FIG. 20 is a perspective view of suction and light apparatus showing an alternative embodiment having a unitary body.

FIG. 21 is a perspective view of suction and light apparatus similar to FIG. 20 but further includes controllable ON/OFF switches for light and suction.

### DETAILED DESCRIPTION OF THE INVENTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

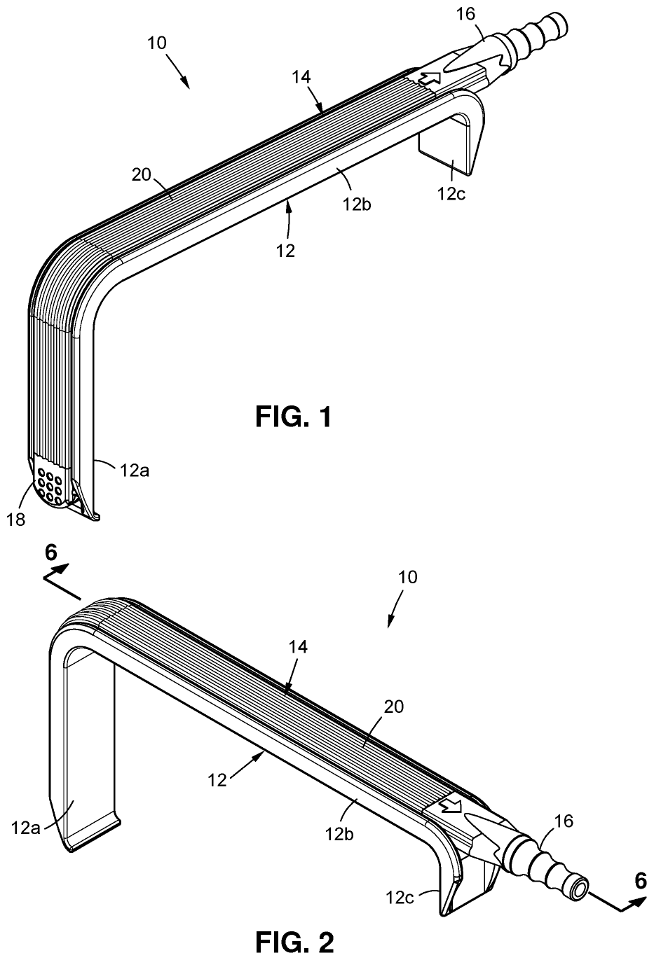

FIG. 1-3 illustrates a surgical retractor assembly 10 which includes a retractor 12 with an adaptable suction tubing apparatus 14. The suction tubing comprises a suction tip 18, an extruded rectangular tubing 20, and an end suction connector 16.

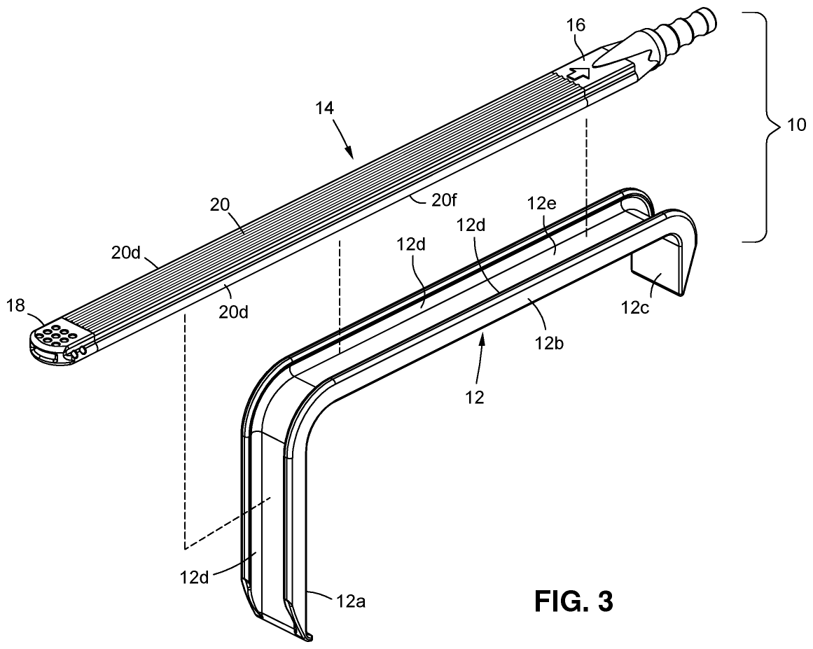

The adaptable suction tubing apparatus 14 is made of flexible polymer material such as PVC, or silicone and placed right over the top surface 12e of the surgical retractor 12 in which conform to the contour of the top surface from front blade 12a portion to midsection body 12b and to the end blade 12c portion. Furthermore the retractor is having inner side channels 12d or grooves for receiving tubing apparatus 14 for via its side protrusion 18e, 20d, 16e to snug fit within the retractor 12 as best seen in FIG. 3.

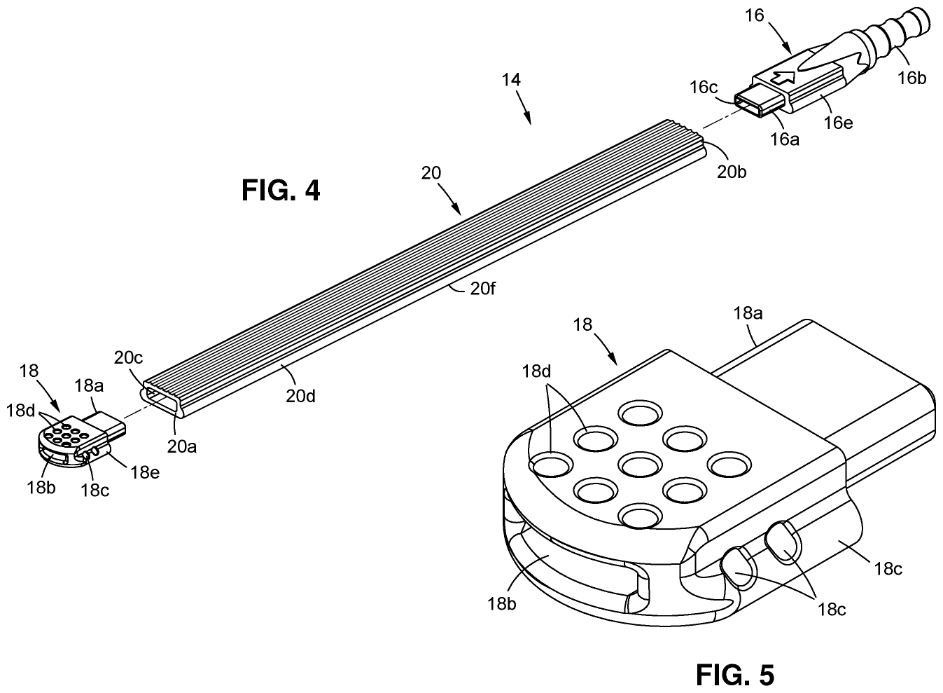

FIG. 4 is showing an exploded view of adaptable tubing apparatus 14. The extruded rectangular tubing 20 having distal end 20a and proximal end 20b. The tubing also includes at least one central suction lumen 20c. A hollow suction tip connector 18 is bonded to distal end 20a and on the other end a hollow end suction connector 16 bonded to proximal end 20b. The end suction connector 16 is made of hard polymer martial and having wall thicknesses. The connector 16 includes a barbed portion 16b for connecting to suction hose at one end, and having mating part 16a at the other end.

The suction tip connector has pluralities of suction ports on top 18d, sides 18c and front 18b, and having a mating part 18a for bounding into the distal end 20a of suction strip. As best seen in FIG. 5.

Furthermore, all the components are having side protrusion 18e, 20d, 16e or bumps for tucking into the receiving channels 12d of the retractor top portion.

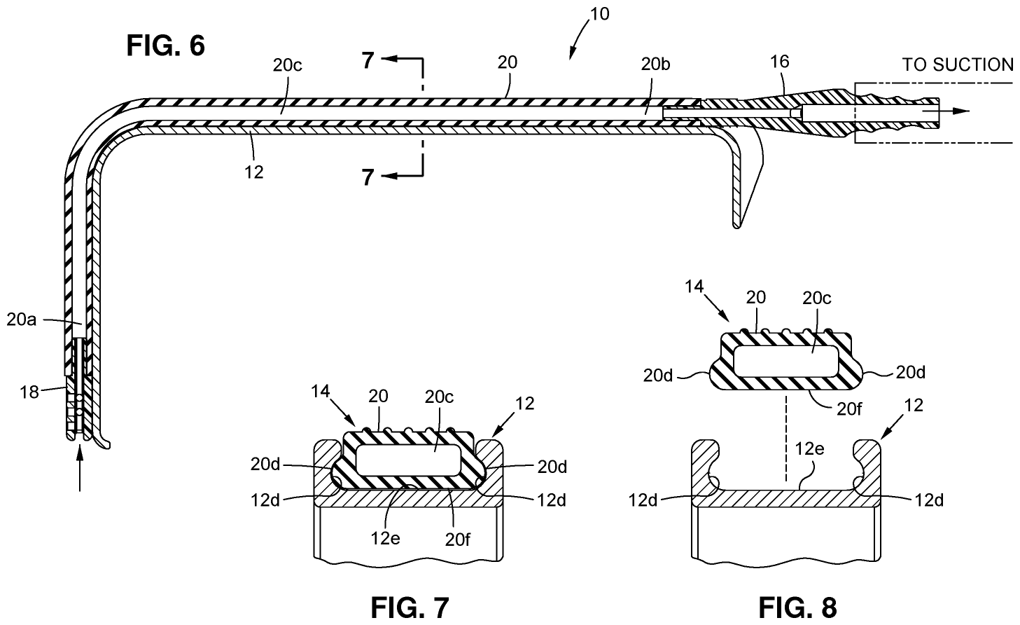

An assembled cross sectional view of present invention is shown in FIG. 6. This view is showing the adaptable suction tubing apparatus 14 has a continuous suction lumen 20c and the device is conform to the contour of a surgical retractor 12 from front blade portion to the end blade portion. FIG. 7-8 are showing fragmentary cross sectional view of being assembled and in exploded state.

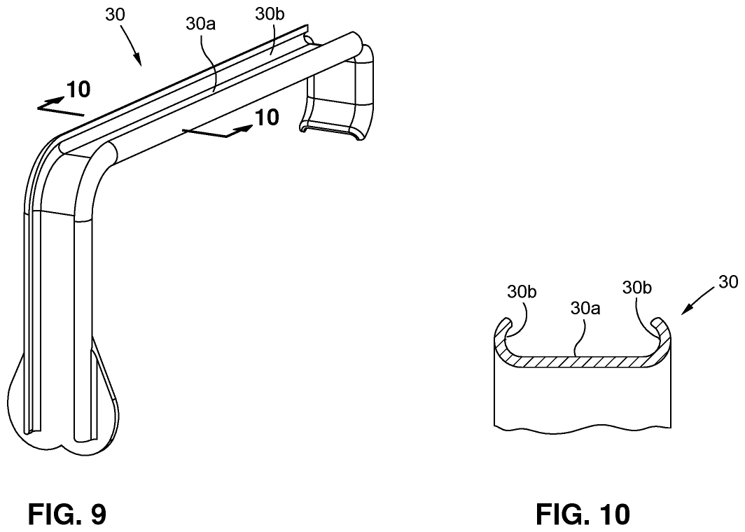

Now referring to FIG. 9-10, a modified version of a surgical retractor 30 is showing the top surface 30a having bended side portions to create side channels 30a for receiving suction tube apparatus 14.

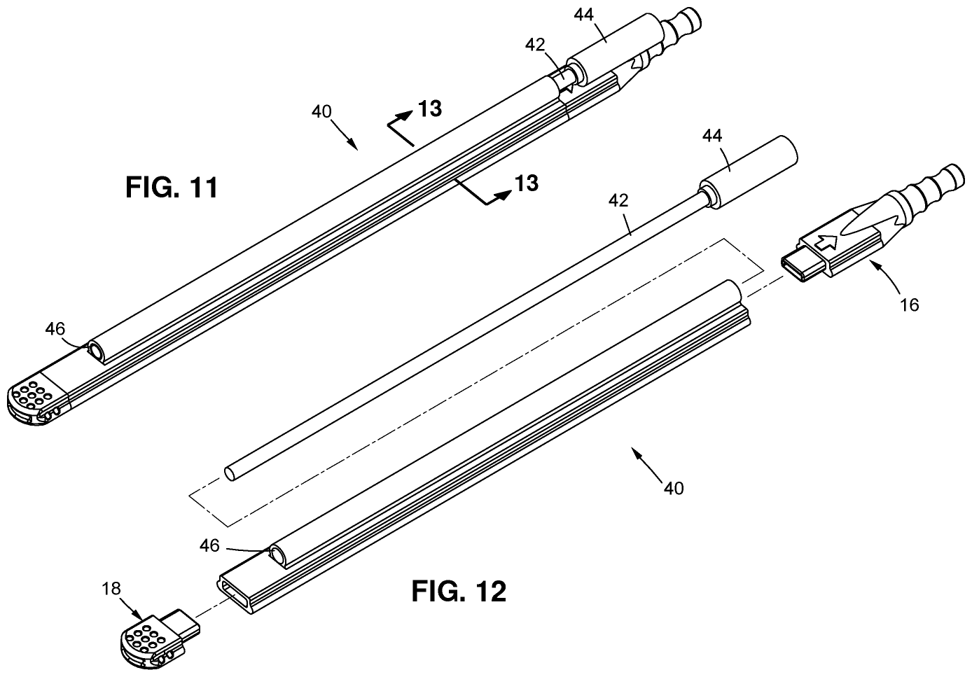

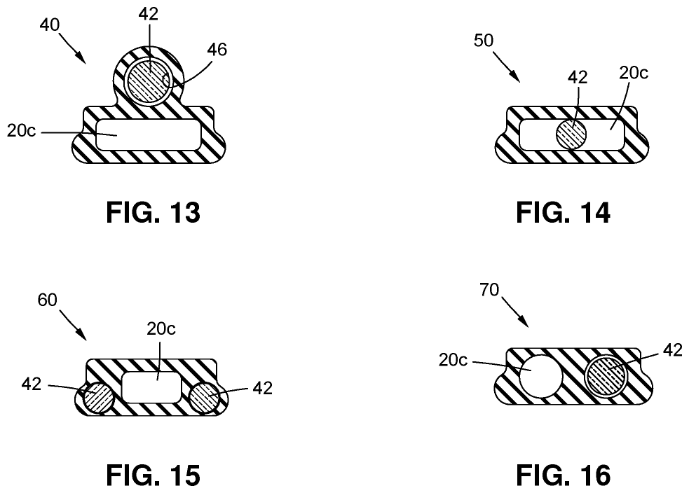

FIG. 11-12 are showing a modified version of adaptable suction tubing apparatus 40 having a fiber optic light on the top portion of suction strip. The fiber optic strands 42 are flexible and conform to device bending as needed to for placement over a surgical retractor. The fiber optic 44 had a universal end connector 44 to receive optical lights from many different light source machines. The fiber optic has end glow light projection known as spot light to view the surgical field for better lighting. The fiber optic 42 also may also have glow body to light up the entire suction device as needed. The fiber optic light has at least one dedicated lumen 46 in suction strip as seen in FIG. 13.

Alternatively, the fiber optic strand 42 can be placed inside suction lumen 20c as shown in FIG. 14 suction assembly 50. FIG. 15 also shows another suction assembly 60 which includes a pair of fiber optic lights 42 adjacent to suction lumen 20c.

Another suction strip embodiment 70 can have two parallel lumens next to each other for both suction 20c and light 42 as seen in FIG. 16.

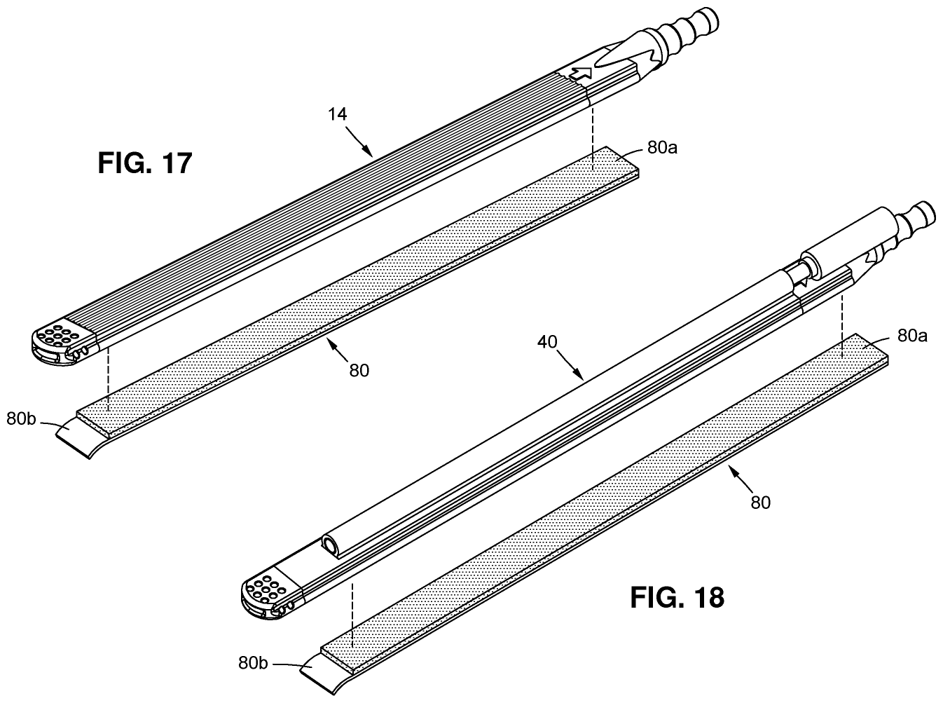

FIG. 17-18 are showing two types of adaptable suction tubing apparatus 14, 40 having double sided adhesive tapes 80 for be use for standard surgical retractor. The adhesive tape 80 is bonded from top side 80a to the bottom side of suction tubing devices and on the bottom side of adhesive, a peel off sheet 80b is placed. The peel off sheet can be removed when the surgeon is ready to place it over the surgical retractor for usage.

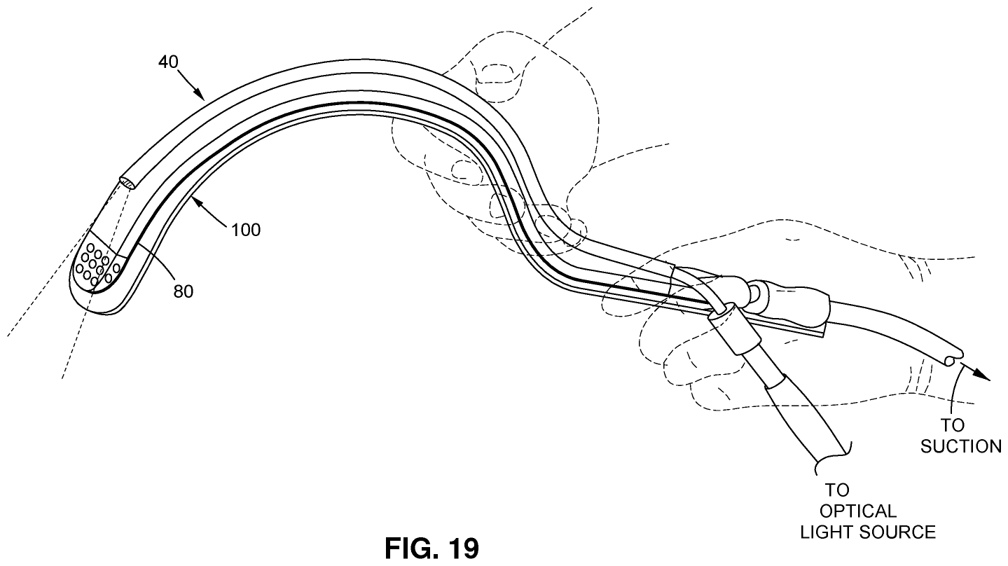

FIG. 19 is showing an adaptable suction tubing apparatus 40 is place over a standard malleable retractor 100 via the double sided tape 80. The malleable retractor is good example to show the invention in used with respect to bending and twisting the malleable reactor 100 in any form and shape. The adaptable suction tubing 40 adapt accordingly.

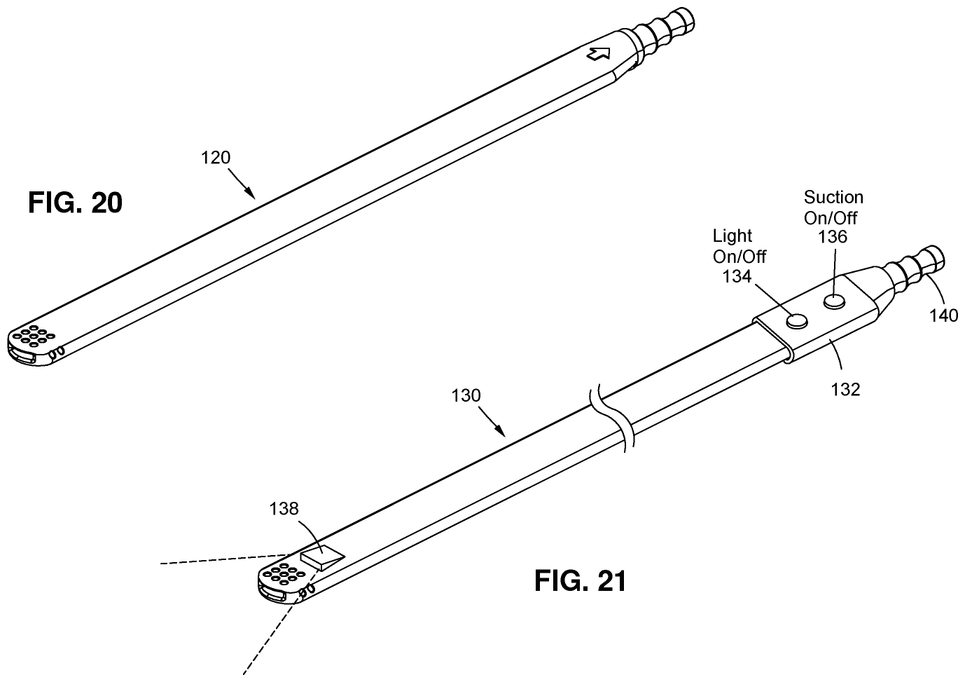

FIG. 20 illustrates an alternative embodiment of an adaptable suction tubing apparatus 120 having a unitary body that includes suction tip, main body and end suction connector. The unitary body is made of one molded piece.

FIG. 21 is illustrating an alternative embodiment of an adaptable suction tubing apparatus 130 similar to FIG. 20 but having a control unit 132 connected to end suction port 140. The control unit having ON/OFF switches for fiber optic light 134 and suction 136. Furthermore, the fiber optic light tip 138 extend out near proximal end to project light at surgical field.

While this invention is susceptible to embodiments in many different forms, this specification and the accompanying drawings disclose only some specific forms as examples of the invention. The invention is not intended to be limited to the embodiments as described; however, the scope of the invention is pointed out in the appended claims.

We claim:

1- An adaptable suction tubing apparatus for a surgical retractor comprises:
an extruded rectangular tubing having distal and proximal ends, said tubing includes at least one lumen for purpose of suctioning, and having protrusions at its sides for mating with a surgical retractor top surface channels, and;
a hollow tip connector, said hollow tip connector having plurality of ports for purpose of evacuation at one end, and at the other end attached to said rectangular tubing at its distal end; and;
a hollow end connector, said end connector connects to external suction source at one end and at the other end attached to said rectangular tubing at its proximal end.

2- An adaptable suction tubing apparatus for a surgical retractor according to claim 1, wherein said protrusions at its sides are rounded in shape.

3- An adaptable suction tubing apparatus for a surgical retractor according to claim 1, wherein said tip connector further includes rounded protrusions at its sides to mate with a surgical retractor top surface channels.

4- An adaptable suction tubing apparatus for a surgical retractor according to claim 1, wherein said end connector further includes rounded protrusions at its sides to mate with a surgical retractor top surface channels.

5- An adaptable suction tubing apparatus for a surgical retractor comprises:
an extruded rectangular tubing having distal and proximal ends, said tubing includes at least one lumen for purpose of suctioning and at least one lumen for purpose of passing fiber optics light, and having protrusions at its sides for mating with a surgical retractor top surface channels, and;
a hollow tip connector, said hollow tip connector having plurality of ports for purpose of evacuation at one end, and at the other end attached to said rectangular tubing at its distal end; and;
a hollow end connector, said end connector connects to external suction source at one end and at the other end attached to said rectangular tubing at its proximal end.

6- An adaptable suction tubing apparatus for a surgical retractor according to claim 5, wherein said at least one lumen for fiber optics light, said lumen can be placed on top of said extruded suction tubing.

7- An adaptable suction tubing apparatus for a surgical retractor according to claim 5, wherein said at least one lumen for fiber optics light, said lumen can be placed adjacent to said suction lumen.

8- An adaptable suction tubing apparatus for a surgical retractor according to claim 5, wherein said at least one lumen for fiber optics light, said lumen can be placed inside said suction lumen.

9- An adaptable suction tubing apparatus for a surgical retractor comprises:
an extruded rectangular tubing having distal, proximal ends, top surface and bottom surface, said tubing includes at least one lumen for purpose of suctioning and at least one lumen for purpose of passing fiber optics light, and;
a hollow tip connector, said hollow tip connector having plurality of ports for purpose of evacuation at one end, and at the other end attached to said rectangular tubing at its distal end; and;
a hollow end connector, said end connector connects to external suction source at one end and at the other end attached to said rectangular tubing at its proximal end, and;
a double sided adhesive tape, said double sided adhesive tape having a top surface and bottom surface, said top surface attached to bottom of said rectangular tubing and said adhesive bottom surface attached to top surface of a surgical retractor.
